(19) United States
(12) Patent Application Publication
(10) Pub. No.: US 2025/0049530 A1
(43) Pub. Date: Feb. 13, 2025

(54) STEREOTACTIC DEVICE FOR USE IN MEDICAL PROCEDURES

(71) Applicant: NESA MEDTECH PRIVATE LIMITED, Bangalore (CN)

(72) Inventors: Suraj Rajan Kanyan KANDI, Pune (IN); Arun Paliakkara JOSE, Thrissur (IN); Usha B R, Bengaluru (IN); Sanjay GIRISH, Bangalore (IN); Vineeshkumar Parameshwaran VISHWAKARMA, Thrissur (IN)

(21) Appl. No.: 18/721,196

(22) PCT Filed: Dec. 19, 2022
(86) PCT No.: PCT/IN2022/051097

§ 371 Date: Jun. 18, 2024

(30) Foreign Application Priority Data
Dec. 19, 2021 (IN) 202141053284

(51) Int. Cl. A61B 90/10; A61B 90/50

(52) U.S. Cl. CPC A61B 90/10; A61B 90/50

(57) ABSTRACT

Embodiments of the present disclosure discloses a stereotactic device (100, 200). The stereotactic device (100, 200) comprises, a clamp (102, 202), a bar (106, 206) configured for being held by the clamp (102, 202), and an articulating arm assembly (120, 220). The articulating arm assembly (120, 220) comprises a bottom arm (122, 222) being coupled to the bar (106, 206) through a first flexible joint (130, 230), a top arm (126, 226) being coupled to the bottom arm (122, 222) through a second flexible joint (140, 240), and being coupled to a sleeve (150, 250) through a third flexible joint (145, 245), wherein the first flexible joint (130, 230), the second flexible joint (140, 240) and the third flexible joint (145, 245) provide three degrees of freedom for moving and positioning a tip (156) of a medical device (154) attached to the sleeve (150, 250).

### TECHNICAL FIELD

The present disclosure generally relates to the field of medical electro-mechanical devices and systems, and more particularly to stereotactic device for conducting procedures that require positioning of therapy devices in a subject's body for a prolonged time.

### BACKGROUND

During any procedure on uterine fibroids, ablation for example, the device for carrying out ablation needs to be inserted, positioned, and supported in the precise location with the precise orientation in the uterus. The device is normally held in the hand of the clinician delivering the therapy and the clinicians often find it challenging to administer the treatment with precision at the region. Thus, there are chances of the device being prematurely withdrawn or moved from the required position and orientation. It is also possible that the tip of the device delivering the treatment, hereinafter referred to as the delivery device comes into contact with parts of the uterus, not intended by the clinicians. This is not safe as it may damage tissue other than the fibroid being ablated. It is, thus, necessary to ensure that the device is accurately positioned and once positioned, is held in place during the entire procedure.

### BRIEF SUMMARY

This summary is provided to introduce a selection of concepts in a simple manner that is further described in the detailed description of the disclosure. This summary is not intended to identify key or essential inventive concepts of the subject matter nor is it intended for determining the scope of the disclosure.

To overcome at least one of the problems mentioned above, there exists a need for stereotactic device for conducting procedures that require positioning therapy devices in a subject's body for a prolonged time.

The present disclosure discloses a stereotactic device for conducting procedures that require positioning therapy devices in a subject's body for a prolonged time. The stereotactic device comprises a clamp configured for mounting the stereotactic device on to a rigid part of an operating table, a bar comprising a first end and a second end, the first end configured for being held by the clamp and the second end is located in a y-z plane substantially at a center of the operating table in the x direction, an articulating arm assembly comprising, a bottom arm having a first end and a second end, the first end being coupled to the second end of the bar through a first flexible joint, a top arm having a first end and a second end, the first end coupled to the second end of the bottom arm through a second flexible joint, for being rotatable in the y-z plane, a sleeve mounted to the second end of the top arm with a third flexible joint and configured for holding the medical device, wherein the first flexible joint the second flexible joint and the third flexible joint provide three degrees of freedom for moving and positioning the tip of the medical device.

### BRIEF DESCRIPTION OF THE FIGURES

The disclosed method and device will be described and explained with additional specificity and detail with the accompanying figures in which:

FIG. 1A shows a stereotactic device for use in a medical procedure in accordance with an embodiment of the present disclosure.

FIG. 1B shows the articulating arm assembly in accordance with an embodiment of the present disclosure.

FIG. 2 shows another exemplary stereotactic device for use in a medical procedure in accordance with an embodiment of the present disclosure.

FIGS. 3A and 3B shows a side view and an isometric view of an exemplary clamp in accordance with an embodiment of the present disclosure.

FIG. 4 shows an exemplary locking mechanism in accordance with an embodiment of the present disclosure.

FIG. 5A shows an arrangement of the locking mechanism, the bottom arm and the first flexible joint for enabling and disabling the movement of the bottom arm in accordance with an embodiment of the present disclosure.

FIG. 5B shows an arrangement of the locking mechanism, the top arm and the second flexible joint for enabling and disabling the movement of the sleeve coupled to the top arm in accordance with an embodiment of the present disclosure.

FIGS. 6A and 6B shows ball socket based flexible joint mechanism in accordance with an embodiment of the present disclosure.

FIG. 7 shows a stereotactic device with adjustable bar in accordance with an embodiment of the present disclosure.

Further, persons skilled in the art to which this disclosure belongs will appreciate that elements in the figures are illustrated for simplicity and may not have been necessarily drawn to scale. Furthermore, in terms of the construction of the joining ring and one or more components of the bearing assembly may have been represented in the figures by conventional symbols, and the figures may show only those specific details that are pertinent to understanding the embodiments of the present disclosure so as not to obscure the figures with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications to the disclosure, and such further applications of the principles of the disclosure as described herein being contemplated as would normally occur to one skilled in the art to which the disclosure relates are deemed to be a part of this disclosure.

It will be understood by those skilled in the art that the foregoing general description and the following detailed description are exemplary and explanatory of the disclosure and are not intended to be restrictive thereof.

In the present disclosure, relational terms such as first and second, and the like, may be used to distinguish one entity from the other, without necessarily implying any actual relationship or order between such entities.

The terms “comprises”, “comprising”, or any other variations thereof, are intended to cover a non-exclusive inclusion, such that a process or method that comprises a list of steps does not include only those steps but may include other steps not expressly listed or inherent to such a process or a method. Similarly, one or more elements or structures or components preceded by “comprises . . . a” does not, without more constraints, preclude the existence of other elements, other structures, other components, additional devices, additional elements, additional structures, or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skills in the art to which this disclosure belongs. The components, methods, and examples provided herein are illustrative only and not intended to be limiting.

Embodiments of the present disclosure will be described below in detail with reference to the accompanying figures.

Embodiments of the present disclosure discloses a stereotactic device for conducting procedures that require positioning of therapy devices in a subject's body for a prolonged time. The therapy device, hereafter referred to as a medical device may be an ablation device such as radiofrequency, drug delivery or any other surgical interventions in gynecology, for example.

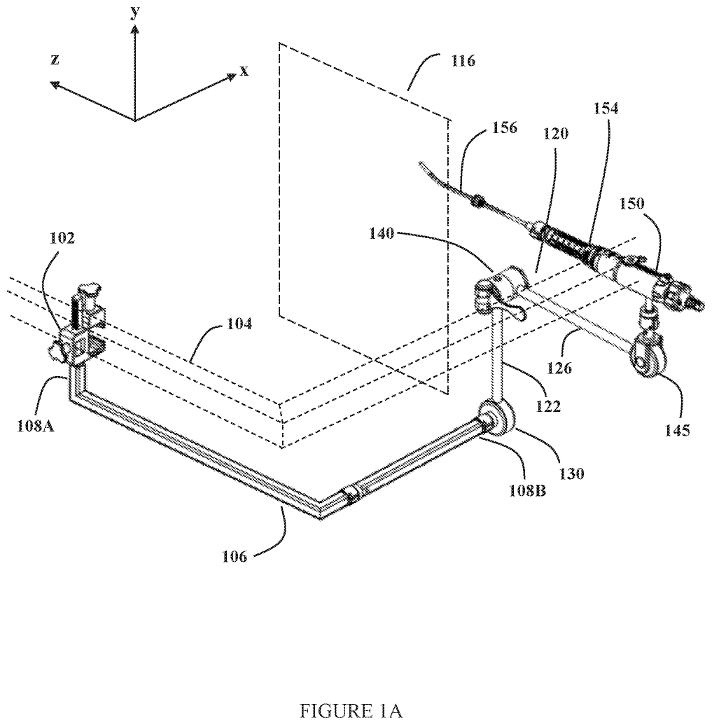

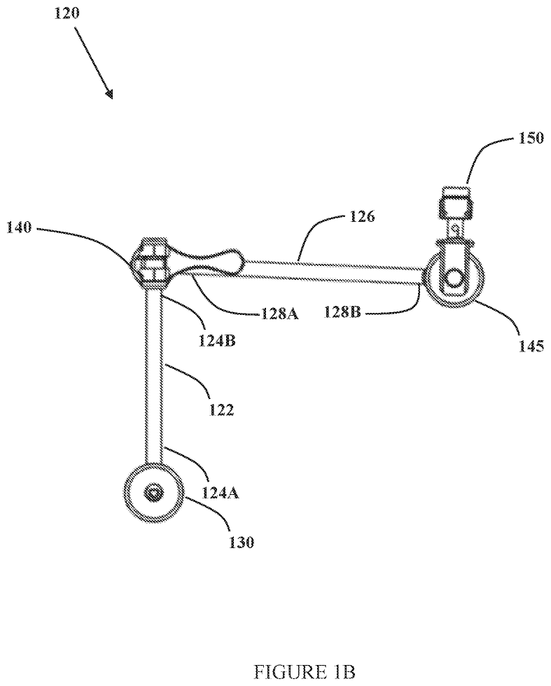

FIG. 1A shows a stereotactic device for use in a medical procedure in accordance with an embodiment of the present disclosure. The stereotactic device disclosed in the present disclosure assists the clinician to position, support, and hold the medical device precisely and steadily in place during the entire procedure. As shown in FIG. 1A, stereotactic device 100 comprises a clamp 102, a bar 106, an articulating arm assembly 120 and a sleeve 150. In one embodiment of the present disclosure, the clamp 102 is configured for mounting the stereotactic device 100 on to a rigid part 104 of an operating table to ensure rigid securing of the entire device without any movement, except those parts that are intended to be moved.

The bar 106 comprises a first end 108A and a second end 108B. The first end 108A is configured for being held by the clamp 102 and the second end 108B is in a y-z plane 116 substantially at a centre of the operating table in the x direction. In an embodiment, bar 106 is square in cross section and is bent at one end to form 108A and bent at another end so its tip reaches the centre of the operating table in the x direction. As described, the bar 106 is square is cross section, but, in other embodiments it may have cylindrical, rectangular, hexagonal section and could either be hollow as well. Once a person skilled in the art has access to the present disclosure, the person may come up with various other alternatives including those for improved structural capabilities all such variants following the principles disclosed here fall under the scope of this disclosure. In one embodiment, the bar 106 extends from the clamp 102 vertically downward for a predetermined length, 150 mm for example, in which 100 mm is available for adjustment of the position at which it is held by clamp 102. This allows a user to adjust at which the medical device 154 held by the disclosed device is located with reference to the top of the operating table on which the subject is located during the procedure. The bar 106 extends up to a predetermined length, 450 mm, for example. The first end 108A of the bar 106 which goes into the clamp 102 may be of square cross section and have linear gear rack which engages with the clamp 102 and prevents the bar 106 from rotating in the axis of linear gear rack while assembled in the clamp 102. The second end of the bar 106 is connected to a first flexible joint 130.

FIG. 1B shows the articulating arm assembly in accordance with an embodiment of the present disclosure. As shown, the articulating arm assembly 120 comprises a bottom arm 122, a top arm 126, and a second flexible joint 140. The bottom arm 122 comprises a first end 124A and a second end 124B, wherein the first end 124A is coupled to the second end 108B of the bar 106 through the first flexible joint 130. The first flexible joint 130 is configured to enable movement of the bottom arm 122 (hence the stereotactic device 100) in both y-z plane and x-y plane.

The top arm 126 comprises a first end 128A and a second end 128B, wherein the first end 128A is coupled to the second end 124B of the bottom arm 122 through the second flexible joint 140. The second flexible joint 140 enables movement of the top arm 126 in y-z plane for adjusting the medical device coupled to the stereotactic device 100. The second end 128B is coupled to a third flexible joint 145 and the third flexible joint 145 connects the second end 128B of the top arm 126 to a sleeve 150, wherein sleeve 150 is configured for holding the medical device 154. Further, the third flexible joint 145 enables movement of the sleeve 150 (hence the medical device 154) in the y-z plane.

As described, the stereotactic device 100 disclosed in the present disclosure assists the clinician to position, support, and hold the medical device precisely and steadily in place during the entire procedure. In one embodiment of the present disclosure, the first flexible joint 130, the second flexible joint 140 and the third flexible joint 145 provide three degrees of freedom for moving and positioning a tip 156 of the medical device 154. In one embodiment of the present disclosure, the second flexible joint 140 has a locking mechanism which locks the first flexible joint 130 and the third flexible joint 145 once the clinician adjusts the desired position of the medical device 154. The way in which the flexible joint works to adjust the desired position and to lock at the desired position is described in detail further below.

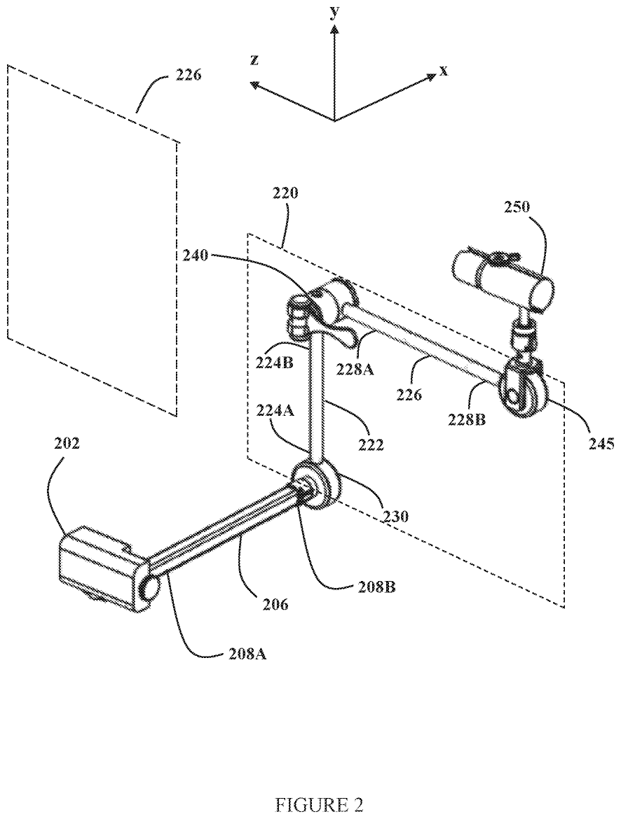

FIG. 2 shows another exemplary stereotactic device for use in a medical procedure in accordance with an embodiment of the present disclosure. The stereotactic device 200 shown in this figure is functionally similar to the stereotactic device 100 shown in FIG. 1. However, the bar 106 is structurally modified to reduce the overall length of the stereotactic device. As shown in FIG. 2, stereotactic device 200 comprises a clamp 202, a bar 206, an articulating arm assembly 220 a sleeve 250. The clamp 202 is configured for mounting the stereotactic device 200 on to a rigid part 104 of an operating table to ensure rigid securing of the entire device without any movement, except those parts that are intended to be moved.

The bar 206 is a straight bar without any bent profile. A first end 208A of the bar 206 which goes into the clamp 202 may be of square cross section and have linear gear rack which engages with the clamp 202 and prevents the bar 106 from rotating while assembled in the clamp 202. A second end of the bar 206 is connected to a first flexible joint 230.

The articulating arm assembly 202 comprises a bottom arm 222, a top arm 223, and a second flexible joint 240. The bottom arm 222 comprises a first end 224A and a second end 224B, wherein the first end 224A is coupled to the second end 208B of the bar 206 through the first flexible joint 230. The first flexible joint 230 enables movement of the bottom arm 222 (hence the stereotactic device 200) in both y-z plane and x-y plane.

The top arm 226 comprises a first end 228A and a second end 228B, wherein the first end 228A is coupled to the second end 224B of the bottom arm 222 through the second flexible joint 240. The second flexible joint 240 enables movement of the top arm 226 in y-z plane for adjusting the medical device coupled to the stereotactic device 200. The second end 228B is coupled to a third flexible joint 245 and the third flexible joint 245 connects the second end 228B of the top arm 226 to a sleeve 250, wherein sleeve 250 is configured for holding the medical device 154. Further, the third flexible joint 245 enables movement of the sleeve 250 (hence the medical device 154) in y-z plane.

In one embodiment of the present disclosure, the first flexible joint 230, the second flexible joint 240 and the third flexible joint 245 provide three degrees of freedom for moving and positioning a tip of the medical device 154 attached to the sleeve 250. In one embodiment of the present disclosure, the second flexible joint 240 has a locking mechanism which locks the first flexible joint 230 and the third flexible joint 245 once the clinician adjusts the medical device 154 at the desired position. The way in which the flexible joint works to adjust the desired position and to lock at the desired position is described in detail further below.

It is to be noted that while describing various components of the stereotactic device, reference is made to the stereotactic device 100 shown in FIG. 1A and FIG. 1B. However, the same is applicable to the stereotactic device 200 shown in FIG. 2, with minor or no modifications.

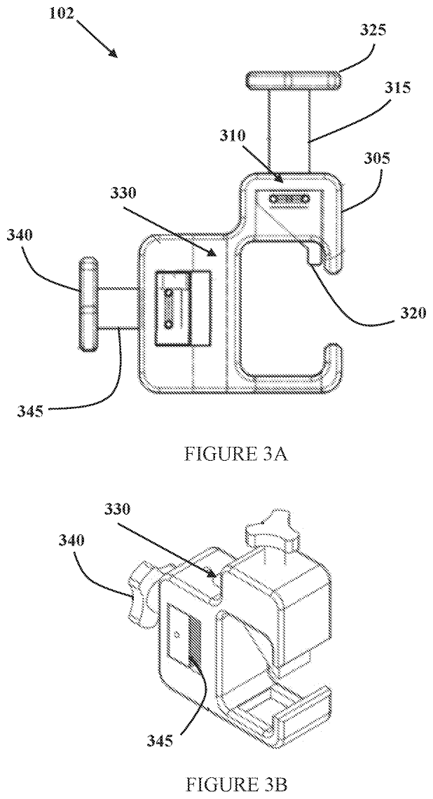

FIGS. 3A and 3B show a side view and an isometric view of an exemplary clamp in accordance with an embodiment of the present disclosure. As shown, the clamp 102 comprises a main body 305 and a first threaded through hole 310 through which a first threaded bolt 315 passes for tightening and loosening the clamp 102. The main body 305 has adequate space between the top and base to accommodate the width rigid part of the operating table. In one embodiment, the first threaded bolt 315 comprises a wedge 320 at one end and a knob 325 at another end for turning the first threaded bolt 315 for tightening or loosening the clamp 102. It is to be noted that the wedge 320 is connected to the threaded bolt 315 with the help of a pin mating with a groove on the threaded bolt 315. This ensures that the wedge 320 does not rotate when the threaded bolt 315 is turned. During the use, the wedge 320 meets the top surface of the rigid part 104 of the operating table and creates a line contact that secures the stereotactic device 100 without any movement. It is however possible that the wedge 320 is a truncated wedge such that it creates a rectangular contact with the top surface of the rigid part 104 of the operating table instead of the line contact described above.

The clamp 102 further comprises a hole 330 for inserting the first end 108A of the bar 106 and a second threaded bolt 335 perpendicular to the hole 330 for tightening the bar 106. The second threaded bolt 335 comprises a knob 340 at one end for turing the bolt and a rack 345 at other end for holding the bar 106, and the rack 345 enables vertical adjustment of the bar 106 for adjusting the medical device 154 along the y axis, when the second threaded bolt 335 is loosened. Further, the linear gear rack of the bar 106 engages with the rack 345 to prevent the movement of the bar 106.

It is to be noted that any other types of clamps may be used for mounting the stereotactic device on to a rigid part of an operating table. However, the clamp 102 disclosed in the present disclosure is designed to adjust the height the stereotactic device and hence the medical device held and positioned by the stereotactic device. Further, the clamp 102 is designed to ensure rigid securing of the entire device without any unwanted movement of the whole, except those parts that are intended to be moved. Even though the embodiment described in detail herein describes mounting the clamp on the frame of the operating table, it may alternatively be mounted on other devices and places, such as an operating table, a system cart, a separate stand created for the purpose, to the floor, an arrangement on the ceiling, with suitable modifications. Suffice to say that a person skilled in the art, having access to this disclosure may make suitable modifications and alterations to mount it firmly to serve the intended purpose within the ambit of normal laboratory modifications and all such modifications, alterations and improvements fall within the scope of this teachings of this disclosure.

As described, the first flexible joint 130, 230, the second flexible joint 140, 240 and the third flexible joint 145, 245 provide three degrees of freedom for moving and positioning a tip of the medical device attached to the sleeve 150. In one embodiment of the present disclosure, the second flexible joint 140 has a locking mechanism which locks not only the second flexible joint 145 with but also the first flexible joint 130 and the third flexible joint 145 with the same action, once the clinician adjusts the location of the medical device 154 at the desired position.

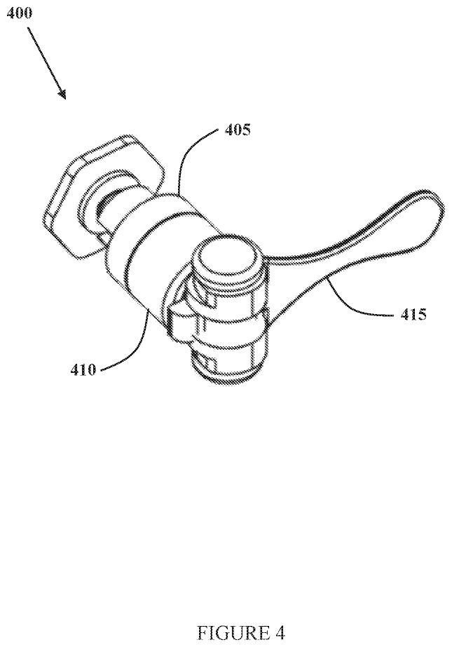

FIG. 4 shows an exemplary locking mechanism in accordance with an embodiment of the present disclosure. As shown, the locking mechanism 400 comprises a first locking profile 405, a second locking profile 410 and a handle 415. The first locking profile 405 connects the bottom arm 122 to the locking mechanism 400, the second locking profile 410 connects the top arm 126 to the locking mechanism 400. The handle is used for engaging the first locking profile 405 with the second locking profile 410 thereby prevents the movement of the articulating arm assembly 120. That is, when locked, the locking mechanism 400 disables the movement of the bottom arm 122 by locking the first flexible joint 130, and also disables the movement of the sleeve 150 by locking the third flexible joint 145.

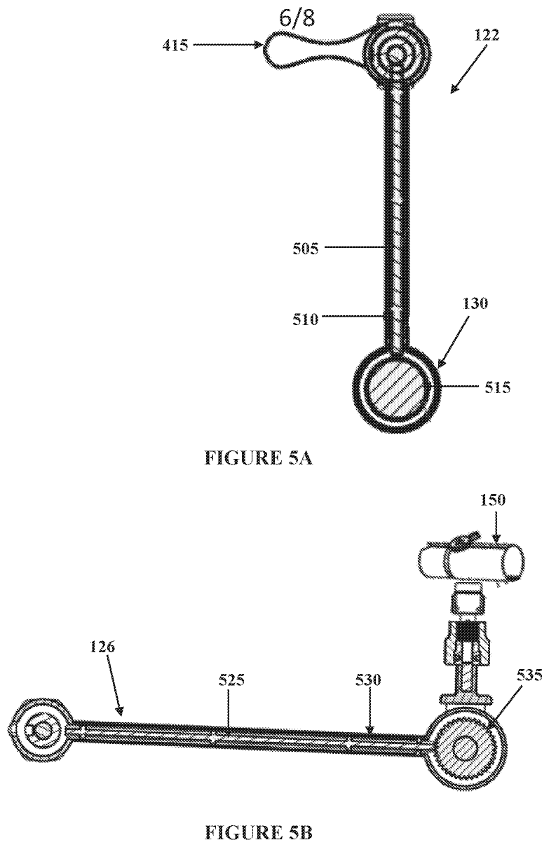

FIG. 5A shows an arrangement of the locking mechanism, the bottom arm and the first flexible joint for enabling and disabling the movement of the bottom arm in accordance with an embodiment of the present disclosure. In one embodiment of the present disclosure, the for enabling and disabling the movement of the bottom arm 122 (for locking and unlocking the articulating arm assembly 120), the bottom arm 122 comprises a shaft 505 and a helical spring 510 around the shaft 505. The position of the shaft 505 is controlled by operating the handle 415 of the locking mechanism 400 for enabling or preventing a rotary movement of the bottom arm 122. In one implementation, the first flexible joint 130 comprises a plurality of teeth 515 inside, and the shaft 505 engages with at least one of the teeth when in the locked position, disabling the movement of the bottom arm 122. When the handle 415 is pushed (for example) the shaft 505 disengages from the teeth thereby enabling the movement of the bottom arm 122.

FIG. 5B shows an arrangement of the locking mechanism, the top arm and the second flexible joint for enabling and disabling the movement of the sleeve coupled to the top arm in accordance with an embodiment of the present disclosure. In one embodiment of the present disclosure, for enabling and disabling the movement of the sleeve 150 coupled to the top arm 126 through the third flexible joint 145, the top arm 122 comprises a shaft 525 and a helical spring 530 around the shaft 525. The position of the shaft 525 is controlled by operating the handle 415 of the locking mechanism 400 for enabling or preventing movement of the sleeve 150 coupled to the top arm 126. In one implementation, the third flexible joint 145 comprises a plurality of teeth 535 inside, and the shaft 525 engages with one of the teeth in locked position, disabling the movement of the sleeve 150 coupled to the top arm 126. When the handle 415 is pushed (for example) the shaft 505 disengages from the teeth thereby enabling the movement of the sleeve 150. It is to be noted that the sleeve 150 is coupled to the second end 128B of the top arm 126 through a connector and the third flexible joint (145). That is, one end of the connector is connected to the sleeve 150 and the other end is connected to the third flexible joint 145.

Even though the embodiment described in detail herein describes a first flexible joint 130 and a third flexible joint 145 having teeth mechanism, any other flexible joint (for example ball socket joint) with suitable locking mechanism may be used for enabling the disabling the movement of the bottom arm 122 and the sleeve 150 connected to the top arm 126.

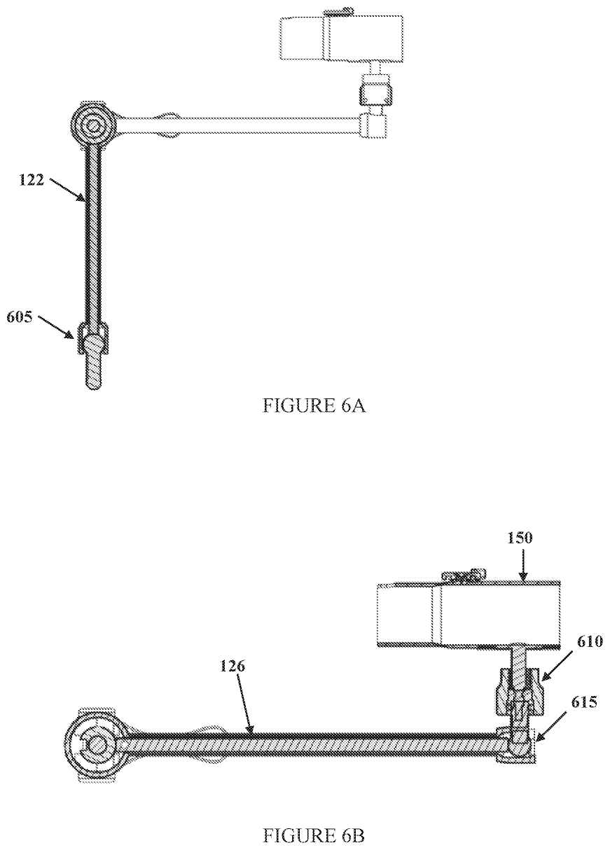

FIGS. 6A and 6B shows ball socket based flexible joint mechanism in accordance with an embodiment of the present disclosure. As shown in FIG. 6A, the bottom arm 122 is coupled to the bar 106 (not shown) through a ball socket joint 605. Referring to FIG. 6B, the top arm 126 and a connector 610 (connecting the top arm 126 and the sleeve 150) are connected thorugh a ball socket joint 605. The ball socket joint 605 enables movement of the bottom arm 122 in y-z and x-y planes. Further, the ball socket joint 615 enables movement of the connector 610 and the sleeve 150 in y-z and x-y planes.

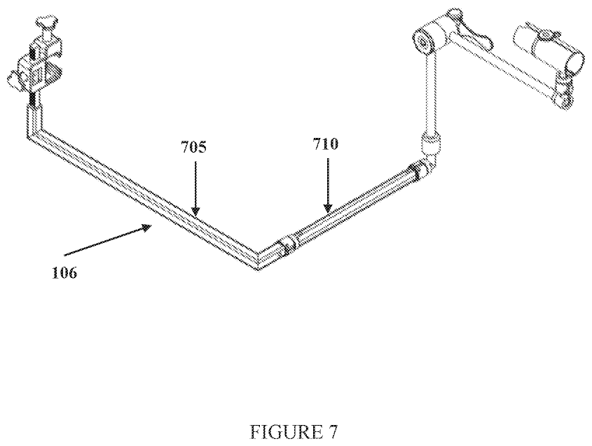

In one embodiment of the present disclosure, the bar 106 is adjustable in length to position the medical device 154 coupled to the stereotactic device 100. FIG. 7 shows a stereotactic device with an adjustable bar in accordance with an embodiment of the present disclosure. To make the bar 106 adjustable, the bar 106 can be made of two or more bars (705 and 710) connected using suitable connectors. In such an implementation, bars having different lengths are provided to replace the bar 710 and hence to acheive desired length. Alternatively, pipe-in-pipe arrangment may be used to adjust the length of the bar 106.

As described, the stereotactic device disclosed in the present disclosure assists the clinician to position, support, and hold the medical device precisely in place during the entire procedure. For example, during ablation of uterus fibroids, the stereotactic device 100 is secured to the operating table using the clamp 102, the first flexible joint 130 and the third flexible joint 145 are unlocked using the handle 415 of the second flexible joint 140 for positioning the bottom arm 122, the top arm 126 and the sleeve 150 and hence to make the tip 156 of the medical device 154 to reach the natural orifice of the subject. Hence, the articulating arm assembly 120 of the stereotactic device 100 device provides three degrees of freedom for moving and positioning the tip (156) of the medical device (154). Once positioned, the articulating arm assembly 120 is locked using the handle 415 of the second flexible joint 140 to keep the medical device 154 in the set position during the procedure. Hence, locking of the stereotactic device 100 is achieved by constraining the relative motion at the three flexible joints by at a singular motion and in this case, it is the second flexible joint 140. In one embodiment of the present disclosure, the bottom arm 122 and the bottom arm 126 are designed with variable length to achieve a range of motions for the device to enter the natural orifice of the subject and later be locked in position parallel to the plane of the operating table.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

We claim:

1. A stereotactic device (100, 200) for use in a medical procedure, the stereotactic device (100, 200) comprising:
a clamp (102, 202) configured for mounting the stereotactic device (100, 200) on to a rigid part (104) of an operating table;
a bar (106, 206) comprising a first end (108A, 208A) and a second end (108B, 208B), the first end (108A, 208A) configured for being held by the clamp (102, 202), and the second end (108B, 208B) is located in a y-z plane (116, 216) substantially at a centre of the operating table in the x direction;
an articulating arm assembly (120, 220) comprising:
a bottom arm (122, 222) having a first end (124A, 224A) and a second end (124B, 224B), the first end (124A, 224A) being coupled to the second end (108B, 208B) of the bar (106, 206) through a first flexible joint (130, 230);
a top arm (126, 226) having a first end (128A, 228A) and a second end (128B, 228B), the first end (128A, 228A) coupled to the second end (124B, 224B) of the bottom arm (122, 224) through a second flexible joint (140, 240), for being rotatable in the y-z plane (116, 216);
a sleeve (150, 250) mounted to the second end (128B, 228B) of the top arm (126, 226) with a third flexible joint (145, 245) and configured for holding the medical device (154);
wherein the first flexible joint (130, 230), the second flexible joint (140, 240) and the third flexible joint (145, 245) provide three degrees of freedom for moving and positioning the tip (156) of the medical device (154).

2. The stereotactic device (100, 200) as claimed in claim 1, wherein the stereotactic device (100, 200) is mountable along the z-axis on the rigid part (104) of the operating table using the clamp (102, 202).

3. The stereotactic device (100, 200) as claimed in claim 1, wherein the flexible joint (140, 240) is a locking mechanism (400).

4. The stereotactic device (100, 200) as claimed in claim 3, wherein the locking mechanism (400) comprises:
a first locking profile (405) which connects the bottom arm (122, 222) to the locking mechanism (400);
a second locking profile (410) which connects the top arm (126, 226) to the locking mechanism (400); and
a handle (415), wherein engaging the handle (415) engages the first locking profile (405) and the second locking profile (410) thereby prevents the movement of the articulating arm assembly (120, 220).

5. The stereotactic device (100, 200) as claimed in claim 1, wherein the bottom arm (122, 222) comprises a shaft (505), wherein a position of the shaft (505) is controlled by operating the handle (415) of the locking mechanism (400) for enabling or preventing a rotary movement of the bottom arm (122, 222) in y-z plane.

6. The stereotactic device (100, 200) as claimed in claim 5, wherein the shaft (505) engages with the flexible joint (130) for preventing rotary movement of the bottom arm (122, 222) in the y-z plane.

7. The stereotactic device (100, 200) as claimed in claim 1, wherein the top arm (126, 226) comprises, a shaft (525), wherein a position of the shaft (525) is controlled by operating the handle (514) of the locking mechanism (400) for enabling or preventing a movement of the medical device (154) in y-z plane.

8. The stereotactic device (100, 200) as claimed in claim 7, wherein the shaft (525) engages with the flexible joint (145) for adjusting angle of the medical device (154) and for preventing movement of the medical device (154).
